(19) United States
(12) Patent Application Publication
(10) Pub. No.: US 2001/0041917 A1
(43) Pub. Date: Nov. 15, 2001

(54) Placebo apparatus for continuous pulse, non-modulated non-burst mode nerve stimulator

(76) Inventor: Robert R. Holcomb, Nashville, TN (US)

Correspondence Address: GARVEY SMITH NEHRBASS & DOODY, LLC, THREE LAKEWAY CENTER, 3838 NORTH CAUSEWAY BLVD., SUITE 3290, METAIRIE, LA, 70002

(21) Appl. No.: 09/756,474

(22) Filed: Jan. 8, 2001

Related U.S. Application Data
(60) Provisional application No. 60/174,891, filed on Jan. 7, 2000.

(51) Int. Cl. A61N 2/00

(52) U.S. Cl. 607/3

(57) ABSTRACT

A placebo device for controlled testing of an apparatus for treating pain with a combination magnetic and electric therapies features a multi-polar, three dimensional magnetic flux field gradient with alternating polarity poles and a plural electrode system, arranged adjacent the magnetic poles, supplied with power to modulate C-fiber activity in the nerves adjacent the treatment area. The placebo device looks like the active device, has the same weight and feel, allows the subject to verify that it is in some sense a magnetic device, however, the device has no physiological action.

### CROSS-REFERENCE TO RELATED APPLICATIONS

Priority of U.S. Provisional Patent Application Serial No. 60/174,891, filed Jan. 7, 2000, incorporated herein by reference, is hereby claimed.

### STATEMENT REGARDING FEDERALLY SPONSORED RESEARCH OR DEVELOPMENT

Not applicable

### REFERENCE TO A “MICROFICHE APPENDIX”

Not applicable

### BACKGROUND OF THE INVENTION

### Field of the Invention

The present invention relates to the field of medical electronics and more particularly to a placebo apparatus for controlled studies of the effectiveness of pain reduction with magnetic devices. As explained below there exist apparatuses for treating human pain by application of an electrical stimulus with the proper current density to the body surface and the response modulated by a magnetic field to allow manipulation of the firing rate of peripheral neurons of the A-fiber and C-fiber nociceptors such that chronic and acute pain may be consistently controlled without discomfort from the stimulation. U.S. Pat. Nos. 5,941,902 (issued Aug. 24, 1999) and 5,312,321 (issued May 17, 1994) both are incorporated herein by reference. The present invention is intended to be used in conjunction with such devices (hereinafter called “active devices” or “treatment devices”) in performing controlled studies of their effectiveness.

### BRIEF SUMMARY OF THE INVENTION

The apparatus of the present invention solves the problems confronted in the art in a simple and straightforward manner. What is provided is a magnetic placebo device which looks like the active device, has the same weight and feel, allows the subject to verify that it is in some sense a magnetic device (the non-treatment side should stick to a metallic surface, attract other similar devices or ferrous objects), however, the treatment side of the placebo device has no physiologic action. The devices described below achieve this goal by imposing a relatively thick layer of high permeability material between a small but powerful magnet, preferably NeFeB. Additionally, the high-permeability material is extended beyond the magnet in the lateral plane to allow a preferred return path for magnetic flux lines, thereby reducing the induction in the tissue below the device. The background of magnetic devices used for the treatment of pain is generally discussed below.

Maurer, et., al., 1994 (U.S. Pat No. 4,431,002) indicates that it is well known that pain can be alleviated by electrical pulses applied to the surface of the body or to electrodes implanted within the body. His invention revealed a transcutaneous electrical nerve stimulation apparatus in which the stimulus pulses are modulated in both time and intensity in a prescribed manner, the pulse amplitude and width decreasing, while the pulse repetition rate increases and vice versa. The advantage of this arrangement is said to produce a comfortable and pleasant sensation at levels sufficient to produce muscle contraction and stimulation of deep afferent nerves to cause the release of endogenous opiates, such as endorphins, which are thought to suppress pain.

Deyo, et., al., (NEJM) concluded that Transcutaneous Electrical Nerve Stimulation (TENS) in patients with chronic low back pain is no more effective than treatment with a placebo, and TENS adds no apparent benefit to that of exercise alone. It is apparent that such studies are done without the proper application and use of the technology. It is further apparent that technology is needed that is easier to understand and use by the operator.

The reduction of efficacy of a C-fiber input by coactivation of mechanoceptive A-fibers is the principle underlying transcutaneous electrical nerve stimulation (TENS). The mechanism involved is referred to as the “Gate Control Theory of Pain Perception” (See FIG. 5). TENS involves electrical activation of mechanoceptive fibers. Mechanoceptive A-fibers are activated at lower electrical stimulation intensities than C-fibers, that is, A-fibers have a low threshold. Thus, the mechanoceptive A-fibers can be selectively activated by low intensity electrical stimulation without increasing the firing rate of C-fibers, that is, A-fibers can be selectively activated by low intensity electrical stimulation without increasing the firing rate of C-fibers. As the intensity of stimulation is increased, it is possible to activate both mechanoceptive and nociceptive fibers. Thus, there is a limit to how much stimulation can be applied in order for the current TENS to work. Patients who use TENS devices are fully aware that if they continue to increase the stimulus intensity, they have more pain, rather than less pain. The increasing pain with stimulation is because of C-fiber activation. In some cases, the intensity of stimulation required to achieve pain relief can be reduced simply by repositioning electrodes and reducing the current flux through tissues while still reaching A-fiber threshold. In other cases, it is not possible to achieve pain relief at sufficiently low intensities to selectively activate A-fibers. In these cases, pain may be increased and TENS is said to have failed. In these cases of failure, the information available suggests that TENS failure is largely due to inappropriate electrode placement and insufficient current flow or density at the point of desired stimulation.

Evidence from the literature, clinical observations and isolated neuronal cell preparation data suggest that efficacy of the active device is best obtained by high frequency, continuous stimulation with high current density in the area of stimulation. Pacing of A-fibers along with simultaneous suppression of C-fiber firing provides reliable control of pain syndromes. For the efficacy of the active devices to be realized, a quadripolar array of positive and negative electrodes are arranged in quadrilateral array such that the positive and negative electrodes are in the proper close proximity to one another such that high current density can be obtained in the area of the nerve fiber to be paced. It is a further object of the active device to suppress the firing rates of C-fibers while increasing the rate of A-fibers. This object is accomplished by placing a Magna Bloc.TM. device within the stimulating electrode. Through this methodology, normal firing patterns can be sent to the central nervous system, frequency coded, for a sensation of comfort rather than pain.

The active device consists of 4 electrodes per unit. The electrodes consist of 4 electrodes of alternating polarity and consist of 2 positive poles and 2 negative poles. The positive and negative poles of the electrode head are aligned in substantially a single plane and are oriented in a quadrilateral configuration with positive poles oriented diagonally opposite one another and negative poles oriented diagonally opposite one another. Built into each electrode is a Magna Bloc.TM. device U.S. Pat. No. 5,312,321 (incorporated herein by reference). This active device allows maximal A-fiber stimulation without the discomfort of C-fiber pain and muscle contraction. The Magna Bloc.TM. controls the excitability of neuromuscular units and blocks C-fiber firing.

An object of the present invention is to provide a placebo device to perform controlled studies of the active device through double blind experimentation. The placebo device should look and feel like the active device, however, the treatment side of the placebo should have no physiologic action.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements and wherein:

FIG. 1 includes two charts diagraming the relationship between flux density and radius;

FIG. 2 illustrates absolute flux density around the placebo device for various designs;

FIG. 3 is a perspective view of the active device electrodes according to one embodiment;

FIG. 4 depicts useful locations for the placement of the electrodes of the active device;

FIG. 5 depicts in graphic form field intensity of the magnetic quadripolar portion of the electrodes of the active device, as determined by scanning in a systematic parallel plane 0.3 cm above the surface of the Magna Bloc.TM. device;

FIG. 6 is a schematic diagram of the placebo device according to one embodiment;

FIG. 7 is a schematic diagram of the placebo device according to a second embodiment; and

FIGS. 8-11 are plots of magnetic fields and densities for various designs of the placebo device.

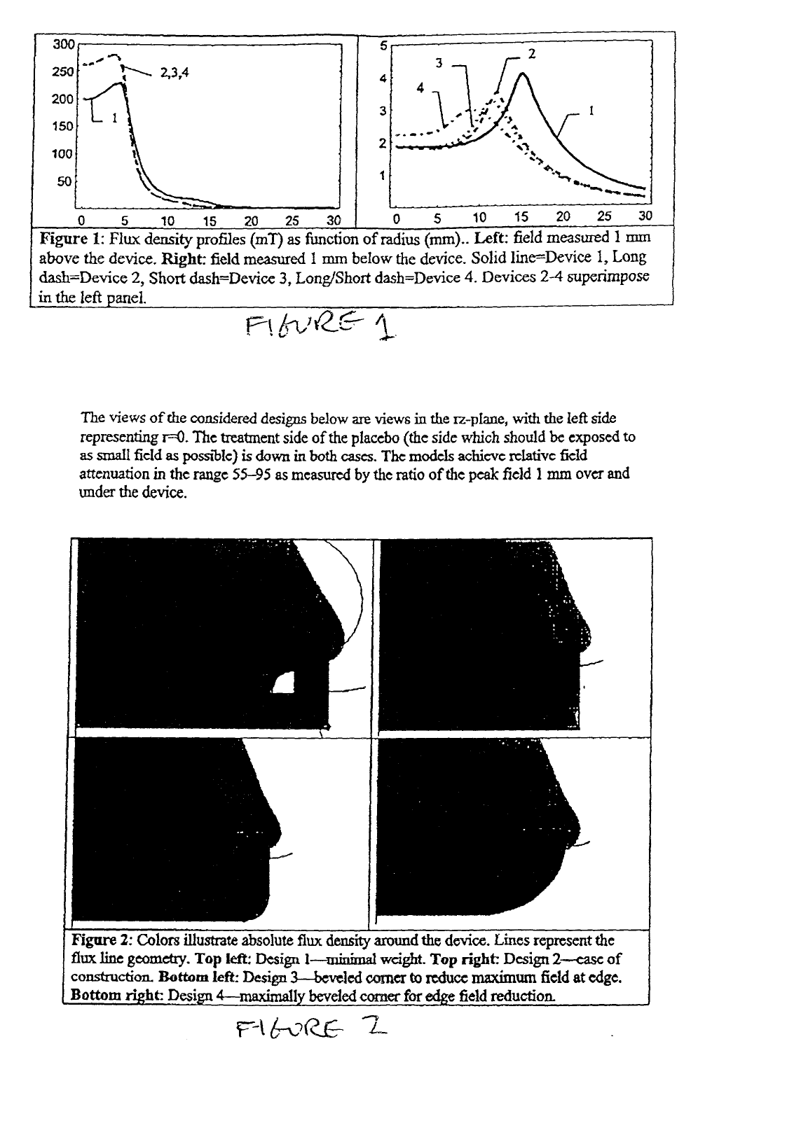

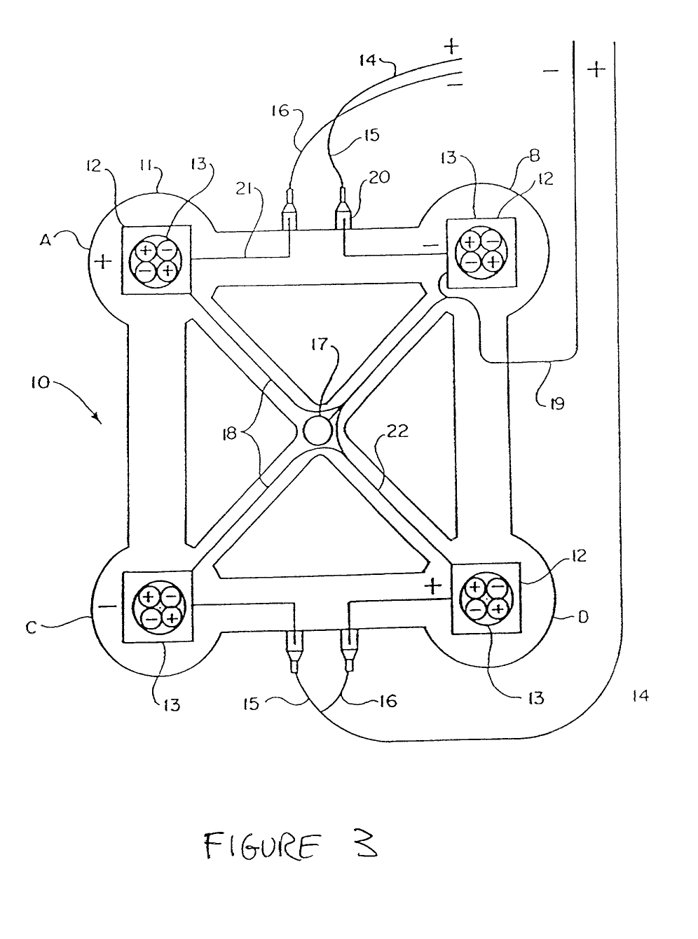

### DETAILED DESCRIPTION OF THE INVENTION

The Dec. 14, 1999 report by Stephan Engstrom is attached and incorporated herein.

Reference will now be made in detail to the presently preferred embodiments of the invention, examples of which are illustrated in the accompanying drawings. Throughout the drawings, like reference characters are used to designate like elements.

Similar placebo models were calculated with μ-metal instead of 1018 stainless steel that is used throughout for the shielding in the models below, but at these field strengths there is no significant difference. The material used for shielding the fields should be highly permeable, but the ability to obtain and work with it can be allowed to influence the specific material used in manufacture.

Four basic designs (1-4) are considered, each addressing a particular concern of the field design.

Design 1: A light weight model with minimal mass in permeable material. The geometry is designed for high flux return and minimal leak field on the bottom side of the device (see FIG. 6).

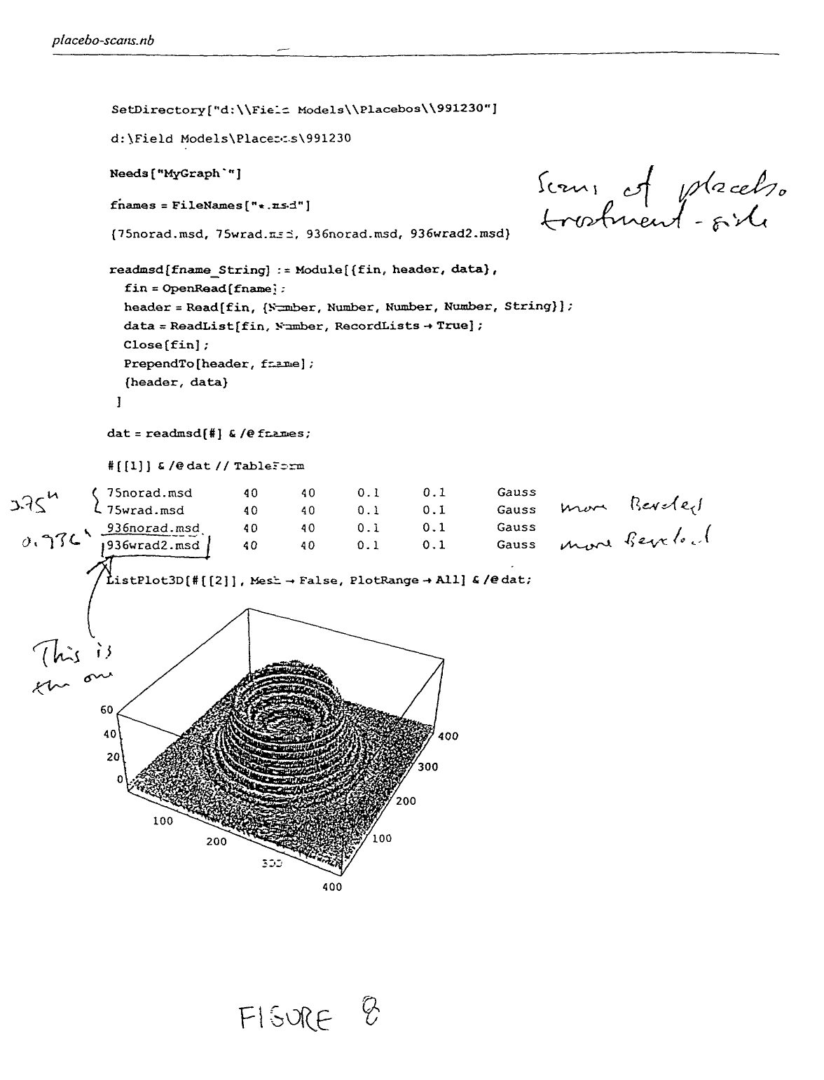

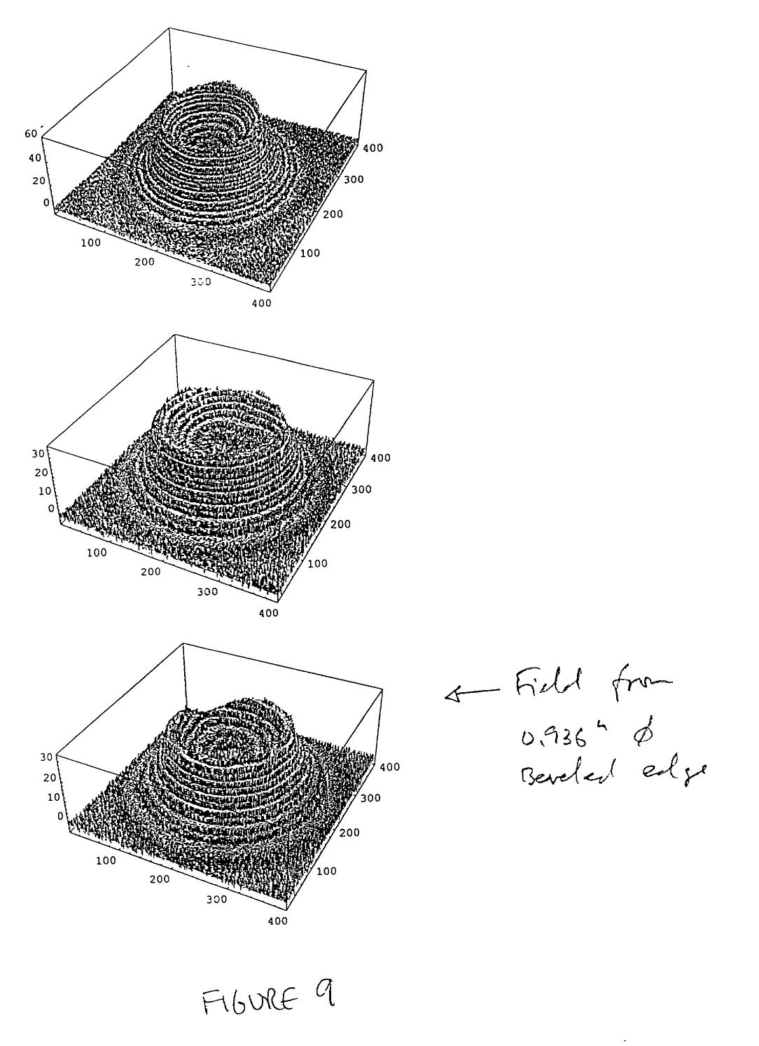

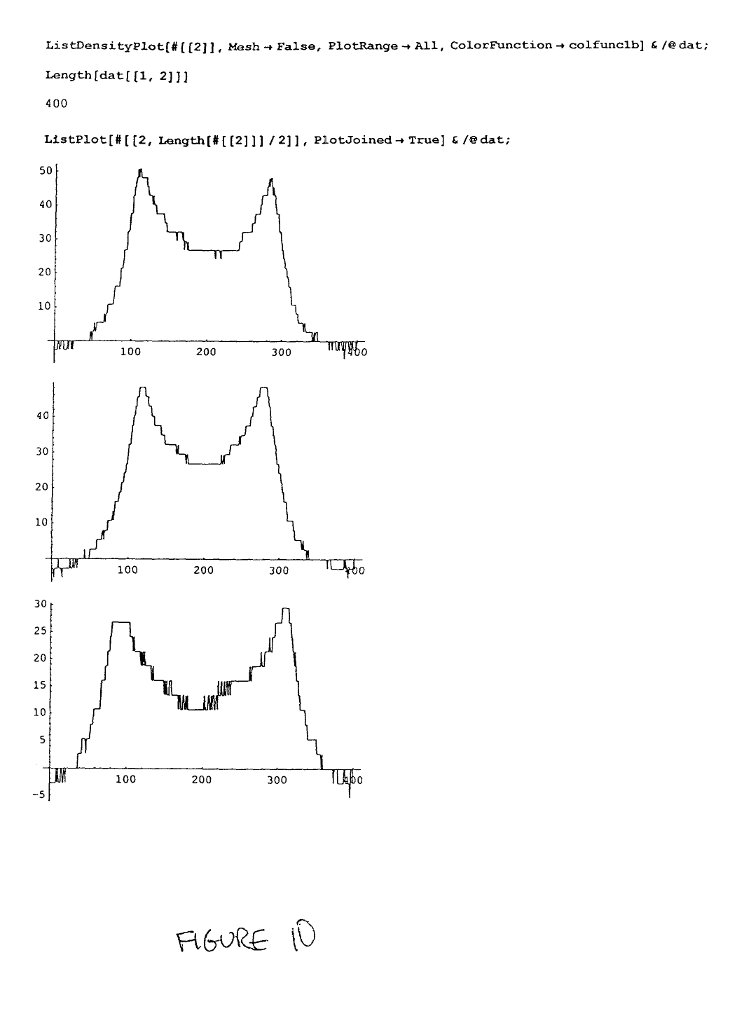

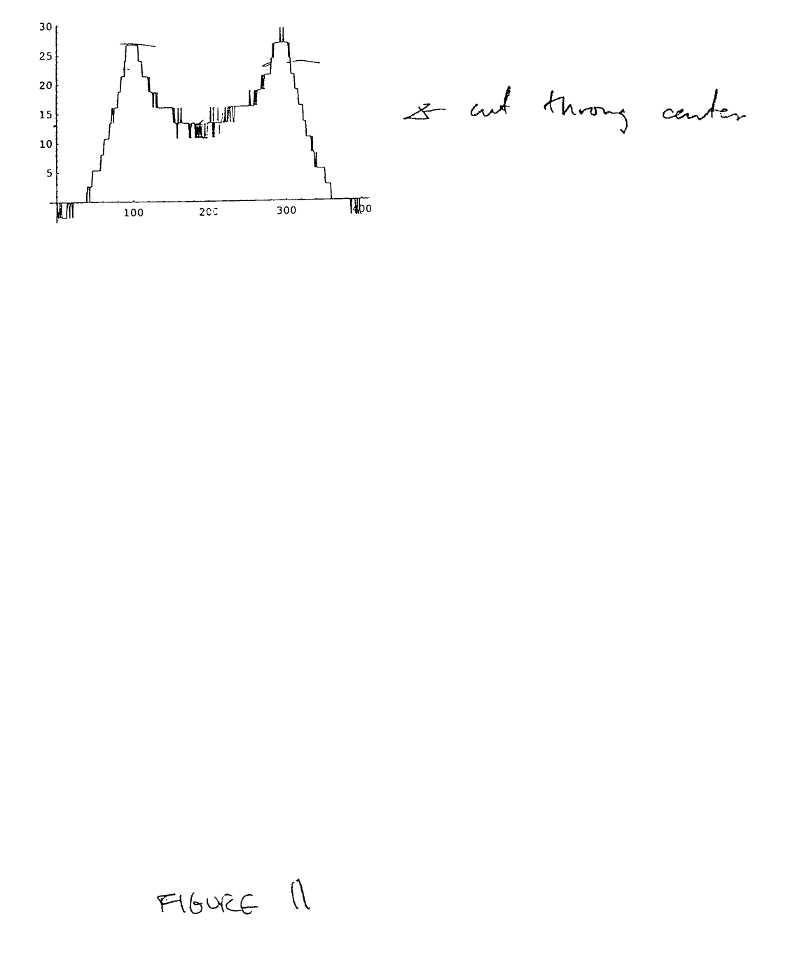

Design 2: An easy to construct placebo with a large field suppression ratio (see FIG. 7).

Design 3: The maximum field and field gradient on the underside of the device is associated with the outer radial edge. This edge-effect can be reduced by using a beveled edge as demonstrated below (see FIG. 7—design 3 is formed from design 2 by rounding off the bottom corner by 1.5 mm).

Design 4: Going to the extremes in terms of rounding off the edges of the device lowers the maximum field on the underside, but instead a larger baseline field value is observed (see FIG. 7—design 4 is formed from design 2 by rounding off the bottom corner by 5 mm).

The views of the considered designs are views in the rz-plane, with the left side representing r=0. The treatment side of the placebo (the side which should be exposed to as small a field as possible) is down in both cases. The models achieve relative field attenuation in the range of 55-95 as measured by the ratio of the peak field 1 mm over and under the device.

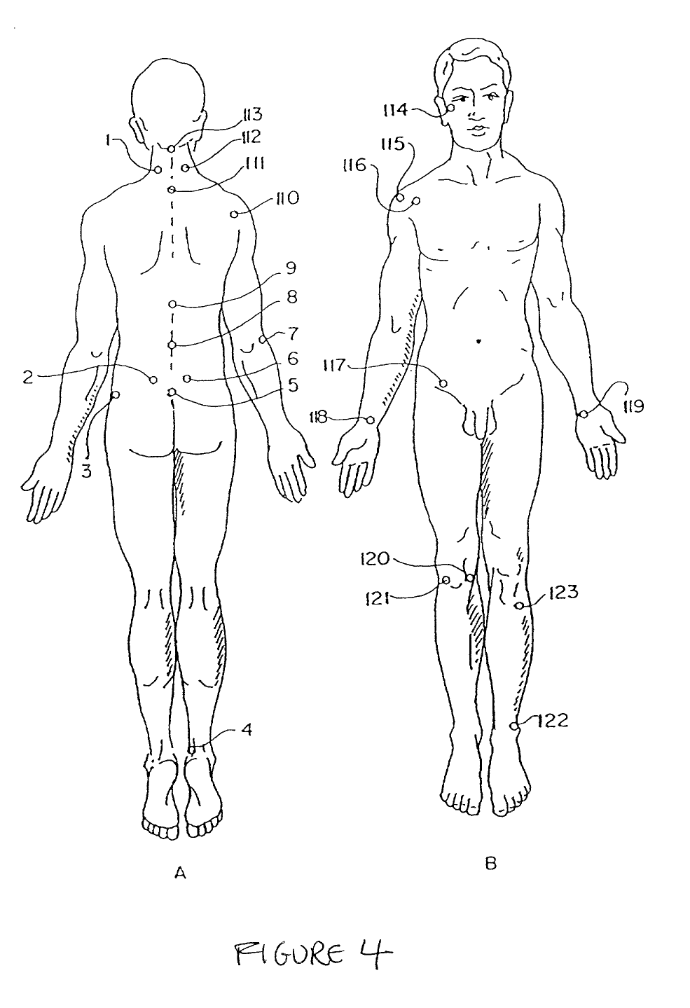

The electrode complex of the active device is schematically illustrated in FIG. 3. Treatment device electrode 10 includes an adhesive means 11 for holding the electrodes 12 and the Magna Bloc.TM. Devices 13 in contact with the human body. Electrode 12 is preferably comprised of 4 electrodes, 2 of which are positive, 2 of which are negative and all of which are electrodes defining opposite diagonal vertices of the quadrilateral shape. Each electrode pad contains a Magna Bloc.TM. which snaps in position.

As embodied herein, Magna Bloc.TM. 13 (magnetic flux generator) comprises four substantially identical magnetic poles held in a plastic containment means that will hold the magnetic bodies in the desired configuration (see U.S. Pat. No. 5,312,321) and which produces a 60.degree. to 70.degree. gradient in the “z” axis (see FIG. 6). The gradient is the slope of the field intensity change over distance.

The active device further contains conducting wires 15 and 16 which connect to electrode wires 21 through connectors 20. The conducting wires 15 and 16 are contained in conducting cable 14. Further embodied in the active device is voltage sensor 17 with electrode connector cables 22 which are ultimately housed in conductor cable 19.

The beneficial effects of the active device are brought about by the ability of the system to maintain a proper current density or flow between the electrodes on a continuous basis in the area of the A-fibers and C-fibers involved in the pain syndrome under treatment. The desired current density is maintained by the electrode pads 12 which are controlled by range monitor (within the housing) and alarm system. The intensity of the current flow will be dictated by a voltage sensor 17. The current flow will alternate every 2 seconds in electrodes B to A, C to D, C to A and B to D. The density of current flow can be operated at a much higher level than in the classic TENS due to the placement of the Magna Bloc.TM. device 13 within the electrode 12. The Magna Bloc.TM. 13 completely relieves the discomfort of C-fiber firing when the C-fiber threshold is exceeded. The Magna Bloc.TM. 13 blocks C-fiber firing, therefore giving a favorable balance to A-fiber/C-fiber ratio and therefore makes this device very effective in relieving pain (see position suggestions for treatment in FIG. 5). For the Magna Bloc.TM. to control C-fiber firing it must have a field gradient of >45.degree.<90.degree. in the “z” axis.

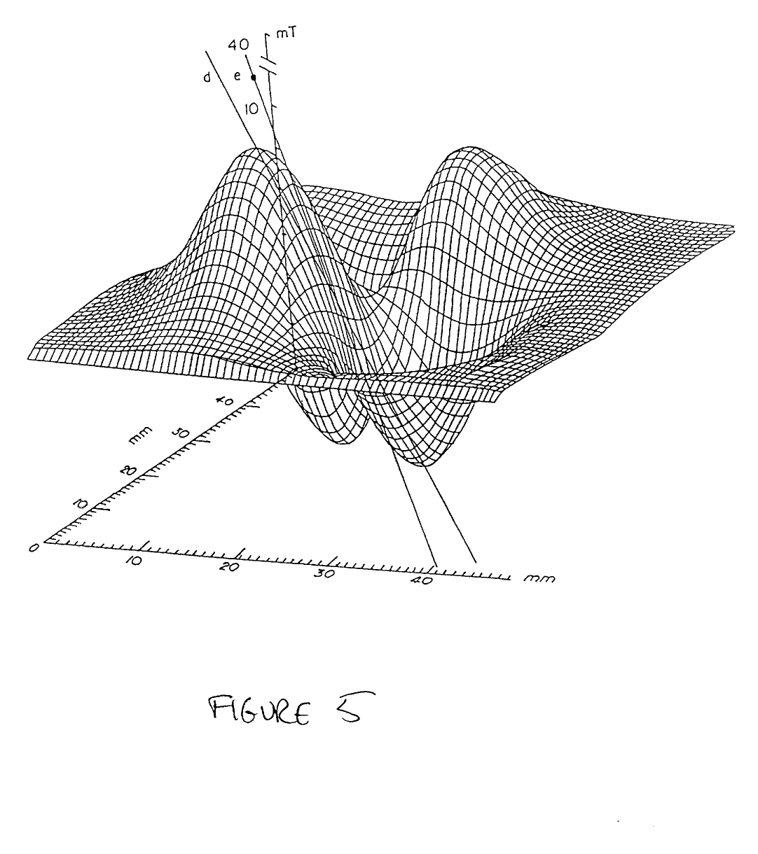

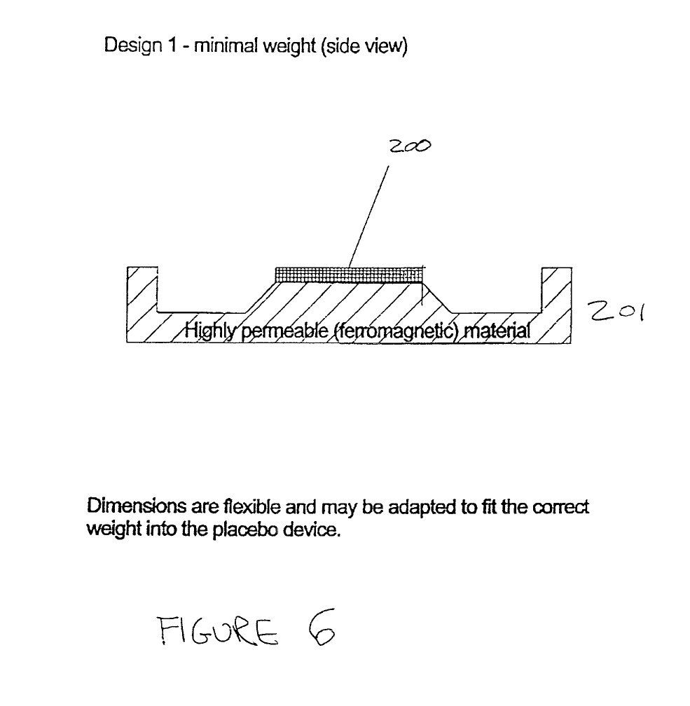

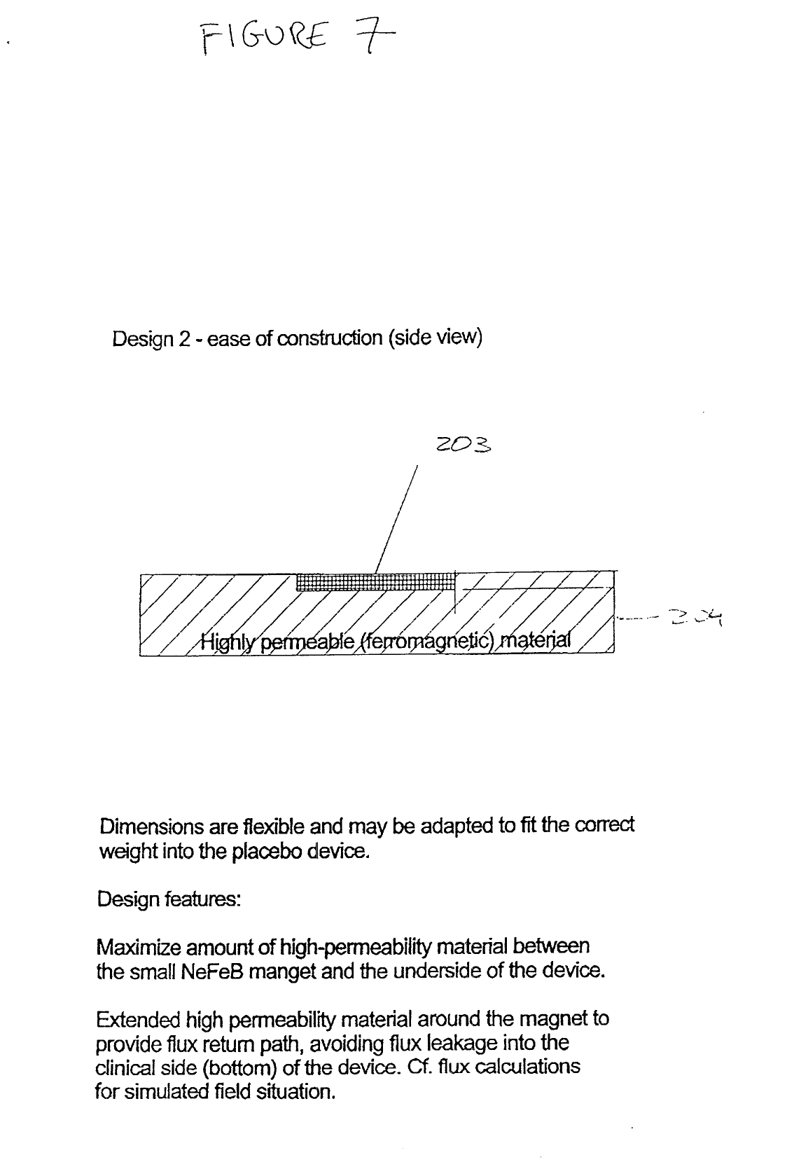

FIG. 4 shows the active device placed at various locations. The placebo device can replace the active device at any location.

Design 1: A light weight model with minimal mass in permeable material. The geometry is designed for high flux return and minimal leak field on the bottom side of the device (see FIG. 6). The device consists of a magnetic material 200, such as NeFeB, attached to a highly permeable (ferromagnetic) material 201. The placebo device would be constructed to look like the active device, having the same weight and feel.

Design 2: An easy to construct placebo with a large field suppression ratio (see FIG. 7). The device consists of a magnetic material 203, such as NeFeB, attached to a highly permeable (ferromagnetic) material 204. The placebo device would be constructed to look like the active device, having the same weight and feel.

Design 3: is formed from design 2 by rounding off the bottom corner by 1.5 mm. The maximum field and field gradient on the underside of the device is associated with the outer radial edge. This edge-effect can be reduced by using a beveled edge as demonstrated below (see FIG. 7).

Design 4: is formed from design 2 by rounding off the bottom corner by 5 mm. Going to the extremes in terms of rounding off the edges of the device lowers the maximum field on the underside, but instead a larger baseline field value is observed (see FIG. 7).

The placebo device allows the performance of controlled studies regarding the effectiveness of pain reduction with the active device. The active device basically allows consistent results on pain treatment because of the ability to produce symmetric current density which is selective for stimulation of A-fiber and suppression of C-fibers.

The foregoing embodiments are presented by way of example only; the scope of the present invention is to be limited only by the following claims.

1. A system for the controlled study of the effectiveness of a medical electronic apparatus for treating pain by the application of an electrical stimulus, comprising:
a) an electrode complex further comprising four electrodes in contact with the body surface of a human to be treated;
b) each electrode further comprising a plurality of quadrapolar magnetic flux generators having four center charged magnetic poles in alternating polarity for generating a three-dimensional flux field gradient >45°<90° in the “Z” axis;
c) power source to activate the electrical stimulus to each electrode for the purpose of modulating C-fiber activity;
d) at least one placebo device further comprising:
e) a magnet;
f) a layer of high permeability material attached to the magnet; and
g) the high permeability ferromagnetic layer being thicker than the magnet:

2. The apparatus of claim 1, wherein the electrode complex further comprises four individual electrode pads at sufficient distance apart from a person to be treated to allow for the proper current density in the field of stimulation when electrical stimulus is applied thereto.

3. The apparatus of claim 1, wherein the electrode complex includes an adhesive means for holding the electrodes in contact the surface of a human body to be treated.

4. The apparatus of claim 1, wherein each quadrapolar magnetic flux generator defines a means for suppressing C-fiber firing, thereby allowing a favorable ration of Aδ/C-fiber firing and thereby more efficiently blocking peripheral generated pain.

5. A method for performing a controlled study of the effectiveness of a medical electronic apparatus for treating pain by the application of an electrical stimulus, comprising the following steps:
a) providing an electrical complex for securing multiple electrodes in contact with the body surface of a human to be treated;
b) the electrical complex further comprising a plurality of quadrapolar magnetic flux generators having four center charged magnetic poles in alternating polarity for generating a three-dimensional flux field gradient >45°<90° in the “Z” axis;
i) at least one placebo device further comprising:
ii) a magnet;
iii) a layer of high permeability ferromagnetic material attached to the magnet; and
iv) the layer being thicker than the magnet.
c) supplying a power source which activates and generates an electrical stimulus through each of the active (non-placebo) magnetic flux generators; and
d) positioning the electrical complex on the body of the human to be treated.
